(19) United States
(12) Patent Application Publication
(10) Pub. No.: US 2024/0374179 A1
(43) Pub. Date: Nov. 14, 2024

(54) APPARATUS INCLUDING HOUSING, POWER SOURCE, AND COUPLER

(71) Applicant: Senseonics, Incorporated, Germantown, MD (US)

(72) Inventors: Abhi Chavan, Germantown, MD (US); Erman Citirik, Germantown, MD (US)

(73) Assignee: Senseonics, Incorporated, Germantown, MD (US)

(21) Appl. No.: 18/314,368

(22) Filed: May 9, 2023

(51) Int. Cl. A61B 5/145; A61B 5/00; A61B 5/1459; H05K 5/00

(52) U.S. Cl. CPC A61B 5/14546; A61B 5/0031; A61B 5/1459; H05K 5/0086; H05K 5/0095; A61B 2560/0214; A61B 2562/166

(57) ABSTRACT

An apparatus including a housing, circuitry at least partially within the housing, a power source including first and second terminals, first and second electrically conductive leads, and a coupler. The first and second electrically conductive leads may be connected electrically to the first and second terminals, respectively, of the power source. The coupler may be attached to the power source. At least a portion of the housing may extend into the coupler. The coupler may include one or more openings through which the first and second electrically conductive leads are capable of being laser welded to first and second contact pads, respectively, of the circuitry. A method for manufacturing the apparatus may include laser welding the first and second electrically conductive leads to the first and second contact pads, respectively, of the circuitry through the one or more openings of the coupler.

### BACKGROUND

### Field of Invention

The present invention relates generally to apparatuses including circuitry at least partially within a housing and a power source. More particularly, the present invention relates to a coupler including one or more openings through which first and second electrically conductive leads connected electrically to first and second terminals, respectively, of the power source are capable of being laser welded to first and second contact pads, respectively, of the circuitry.

### Discussion of the Background

An implantable sensor that has no charge storage device may rely exclusively on an external device for operational power (e.g., to operate its circuitry for making measurements and conveying the data to the external device). The sensor and the external device may each include an inductive element (e.g., coil). The sensor may receive power from the external device when the external device uses its inductive element to generate an electrodynamic field and the inductive elements of the sensor and external device are magnetically coupled within the electrodynamic field. However, with no internal power source, the sensor is dormant if the sensor is not located in the proximity of the external device (i.e., if the inductive elements of the sensor and the external device are not coupled within the electrodynamic field generated by the external device).

For instance, the sensor having no charge storage device may be implanted in the arm of a human patient, and the sensor may be located in the proximity of the external device when the human patient wears an armband having the external device therein. The sensor would be able to take analyte measurements and convey data to the external device while the patient is wearing the armband, but the sensor would not be able to take analyte measurements while the patient was not wearing the armband (e.g., because the human patient is swimming or showering), and the result would be a gap in analyte measurement information.

Accordingly, there is a need for an improved sensor and methods for using the same that improve the ability of the sensor to take analyte measurements.

### SUMMARY

One aspect of the invention may provide an apparatus including a housing, circuitry, a power source, first and second electrically conductive leads, and a coupler. The circuitry may be at least partially within the housing. The circuitry may include first and second contact pads. The power source may include first and second terminals. The first and second electrically conductive leads may be connected electrically to the first and second terminals, respectively, of the power source. The coupler may be attached to the power source. At least a portion of the housing may extend into the coupler. The coupler may include one or more openings through which the first and second electrically conductive leads are capable of being laser welded to the first and second contact pads, respectively, of the circuitry.

In some aspects, the housing may include one or more openings through which the first and second electrically conductive leads are capable of being laser welded to the first and second contact pads, respectively, of the circuitry. In some aspects, the housing may be a polymethylmethacrylate (PMMA) housing. In some aspects, the housing may be a sleeve.

In some aspects, the power source may be a battery. In some aspects, the battery may be a titanium-cased, hermetically-sealed battery.

In some aspects, the coupler may enclose the first and second terminals of the power source. In some aspects, the coupler may include titanium.

In some aspects, the apparatus may further include an encasement material that encases at least a first portion of the circuitry. In some aspects, the encasement material may include a water-resistant epoxy.

In some aspects, the encasement material may be a first encasement material that encases the first portion of the circuitry, the first portion of the circuitry may not include the first and second contact pads, and the apparatus may further include a second encasement material that encases the first and second electrically conductive leads and a second portion of the circuitry that includes the first and second contact pads. In some aspects, the first and second encasement materials may be different. In some aspects, the first and second encasement materials may be the same. In some aspects, the second encasement material may include a water-resistant epoxy. In some aspects, the first encasement material may fill a first portion of the housing, and the second encasement material may fill the coupler and a second portion of the housing that is not filled by the first encasement material.

In some aspects, the encasement material may encase the circuitry and the first and second electrically conductive leads. In some aspects, the encasement material may fill the housing and the coupler.

In some aspects, the apparatus may further include one or more analyte indicators that cover one or more portions of an exterior surface of the housing. In some aspects, the circuitry may include one or more light sources configured to emit excitation light that reaches the one or more analyte indicators after passing through the encasement material. In some aspects, the circuitry may include one or more photodetectors configured to detect emission light that reaches the one or more photodetectors after being emitted by the one or more analyte indicators and passing through the encasement material.

In some aspects, the apparatus may further include a cap over the one or more openings of the coupler.

Another aspect of the invention may provide a method including inserting at least a portion of a housing into a coupler attached to a power source. Circuitry may be at least partially within the housing, the circuitry may include first and second contact pads, and the power source may include first and second terminals. The method may include laser welding first and second electrically conductive leads to the first and second contact pads, respectively, of the circuitry through one or more openings in the coupler. The first and second electrically conductive leads may be connected electrically to the first and second terminals, respectively, of the power source.

In some aspects, the housing may include one or more openings, and the laser welding of the first and second electrically conductive leads to the first and second contact pads, respectively, of the circuitry may be through the one or more openings in the coupler and the one or more openings in the housing.

In some aspects, the method may further include, before inserting at least the portion of the housing into the coupler, encasing at least a first portion of the circuitry in a first encasement material. In some aspects, the first encasement material may include a water-resistant epoxy.

In some aspects, the first portion of the circuitry may not include the first and second contact pads, and the method may further include, after laser welding the first and second electrically conductive leads to the first and second contact pads, respectively, of the circuitry, encasing the first and second electrically conductive leads and a second portion of the circuitry that includes the first and second contact pads in a second encasement material. In some aspects, the second encasement material may fill the coupler and a second portion of the housing that was not filled by the first encasement material. In some aspects, the first and second encasement materials may be different. In some aspects, the first and second encasement materials may be the same. In some aspects, the second encasement material may include a water-resistant epoxy. In some aspects, encasing the first and second electrically conductive leads and the second portion of the circuitry in the second encasement material may include inserting the second encasement material through the one or more openings in the coupler. In some aspects, encasing the first and second electrically conductive leads and the second portion of the circuitry in the second encasement material may further include inserting the second encasement material through one or more openings in the housing.

In some aspects, the method may further include, after laser welding the first and second electrically conductive leads to the first and second contact pads, respectively, of the circuitry, encasing the circuitry and the first and second electrically conductive leads in an encasement material. In some aspects, the encasement material may include a water-resistant epoxy. In some aspects, the encasement material may fill the coupler and the housing. In some aspects, encasing the circuitry and the first and second electrically conductive leads in the encasement material may include inserting the encasement material through the one or more openings in the coupler. In some aspects, encasing the circuitry and the first and second electrically conductive leads in the encasement material may further include inserting the encasement material through one or more openings in the housing.

In some aspects, the method may further include, after laser welding the first and second electrically conductive leads to the first and second contact pads, placing a cap over the one or more openings of the coupler.

Still another aspect of the invention may provide a coupler including a first end, a second end, and one or more openings. The first end may be configured to be attached to a power source. The second end may be configured for insertion of at least a portion of a housing into the coupler. First and second electrically conductive leads connected electrically to first and second terminals, respectively, of the power source may be capable of being laser welded to first and second contact pads, respectively, of circuitry that is at least partially within the housing through the one or more openings.

In some aspects, the coupler may be configured to enclose the first and second terminals of the power source. In some aspects, the coupler may include titanium.

Yet another aspect of the invention may provide an apparatus including a housing, circuitry at least partially within the housing, and one or more supports. The circuitry may include an antenna and a printed circuit board (PCB). The one or more supports may be attached to and extend from the antenna. The one or more supports may be configured to stiffen the circuitry.

In some aspects, the one or more supports may include a bottom support that is attached to and extends from a bottom surface of the antenna. In some aspects, the circuitry may include one or more capacitors mounted on a bottom surface of the PCB, and the bottom support may be attached to the one or more capacitors mounted on the bottom surface of the PCB.

In some aspects, the one or more supports may include side supports that are attached to and extend from side surfaces of the antenna. In some aspects, the side supports may include at least one side support attached to and extending from a right side surface of the antenna and at least one side support attached to and extending from a left side surface of the antenna. In some aspects, the side supports may be attached to a bottom surface of the PCB. In some aspects, the circuitry may include one or more capacitors mounted on a bottom surface of the PCB, and the side supports are attached to the one or more capacitors mounted on the bottom surface of the PCB.

In some aspects, the one or more supports may include a top support that is attached to and extends from a top surface of the antenna. In some aspects, the circuitry may include one or more capacitors mounted on a top surface of the PCB, and the top support may be attached to the one or more capacitors mounted on the top surface of the PCB.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting aspects of the present invention. In the drawings, like reference numbers indicate identical or functionally similar elements.

FIG. 1 is a schematic view illustrating a system embodying aspects of the present invention.

FIG. 2A is an exploded view of an apparatus of the system according to some aspects of the present invention.

FIG. 2B shows a power source and a coupler of the apparatus according to some aspects of the present invention.

FIG. 2C shows circuitry at least partially within a housing of the apparatus according to some aspects of the present invention.

FIGS. 2D and 2E show a portion of the housing extending into the coupler of the apparatus according to some aspects of the present invention.

FIGS. 2F and 2G show perspective and cross-sectional views of an apparatus including a housing, power source, coupler, and one or more stiffeners according to some aspects of the present invention.

FIG. 3 is a flow chart illustrating a process according to some aspects.

FIG. 4 is a flow chart illustrating a process according to some aspects.

### DETAILED DESCRIPTION

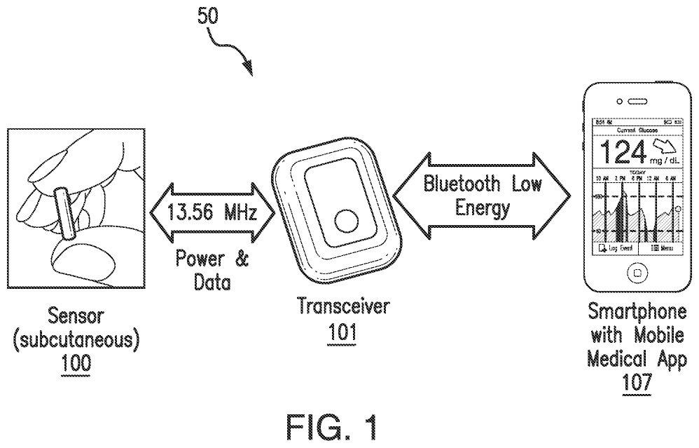

FIG. 1 is a schematic view of an exemplary system 50 embodying aspects of the present invention. In some aspects, the system 50 may be an analyte monitoring system. In some aspects, the system 50 may be a continuous analyte monitoring system (e.g., a continuous glucose monitoring system). In some aspects, the system 50 may include one or more of an apparatus 100, an external device 101, and a display device 105.

In some aspects, the apparatus 100 may be an implantable device. In some aspects, the apparatus 100 may be a wireless implantable device. In some aspects, the apparatus 100 may be a sensor (e.g., an analyte sensor). In some aspects, the apparatus 100 may include one or more optical sensors (e.g., one or more fluorometers). In some aspects, the apparatus 100 may be chemical or biochemical sensors. In some aspects, the apparatus 100 may be a radio frequency identification (RFID) device. In some aspects, the apparatus 100 may be a small, fully subcutaneously implantable sensor that detects the presence, amount, and/or concentration of an analyte (e.g., glucose, oxygen, cardiac markers, low-density lipoprotein (LDL), high-density lipoprotein (HDL), or triglycerides) in a medium (e.g., interstitial fluid) of a living animal (e.g., a living human). However, this is not required, and, in some alternative aspects, the apparatus 100 may be a partially implantable (e.g., transcutaneous) device or a fully external sensor. In addition, although aspects of the invention are described with respect to an analyte monitoring system in which the apparatus 100 is an analyte sensor, this is not required. In some alternative aspects, the apparatus 100 is not a sensor and is instead a different type of apparatus, such as, for example and without limitation, an insulin pump (e.g., an implantable insulin pump), a pacemaker (e.g., an implantable pacemaker), or electrical/heat therapy device (e.g., an implantable electrical/heat therapy device).

In some aspects, the external device 101 may be an externally worn device (e.g., attached via an armband, wristband, waistband, or adhesive patch). In some aspects, the external device 101 may remotely communicate with the apparatus 100 (e.g., via near field communication (NFC)). In some aspects, the external device 101 may communicate with the apparatus 100 to initiate and/or read data (e.g., measurements) from the apparatus 100. In some aspects, the external device 101 may be a transceiver. In some aspects, the external device 101 may be a smartphone (e.g., an NFC-enabled smartphone). In some aspects, the external device 101 may communicate information (e.g., one or more analyte measurements) wirelessly (e.g., via a Bluetooth™ communication standard such as, for example and without limitation Bluetooth Low Energy) to an application running on a display device 105 (e.g., smartphone). In some aspects, the display device 105 may additionally or alternatively communicate directly with the apparatus 100 (e.g., via near field communication (NFC)). In some aspects, the display device 105 may communicate with the apparatus 100 to initiate and/or read data (e.g., measurements) from the apparatus 100.

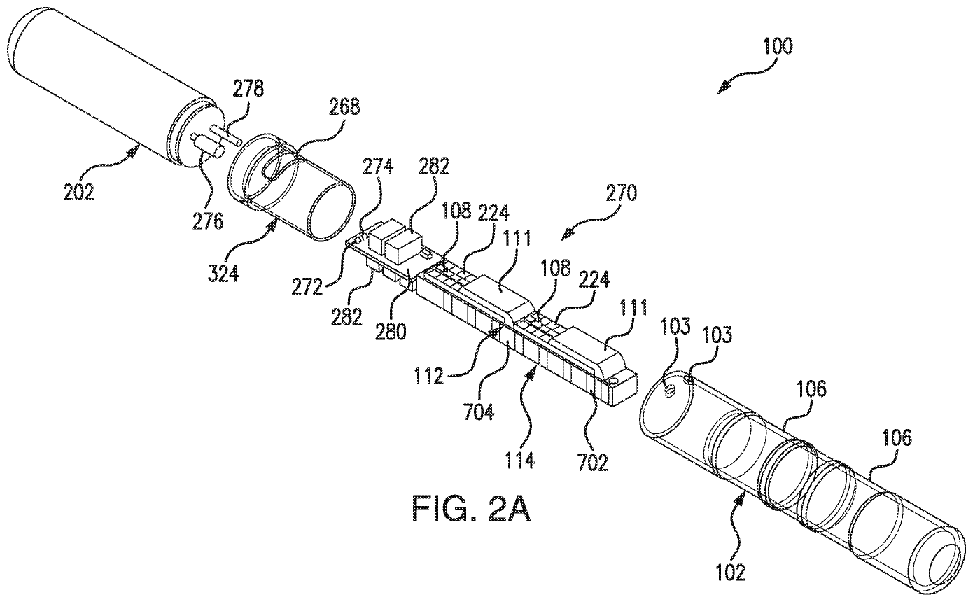

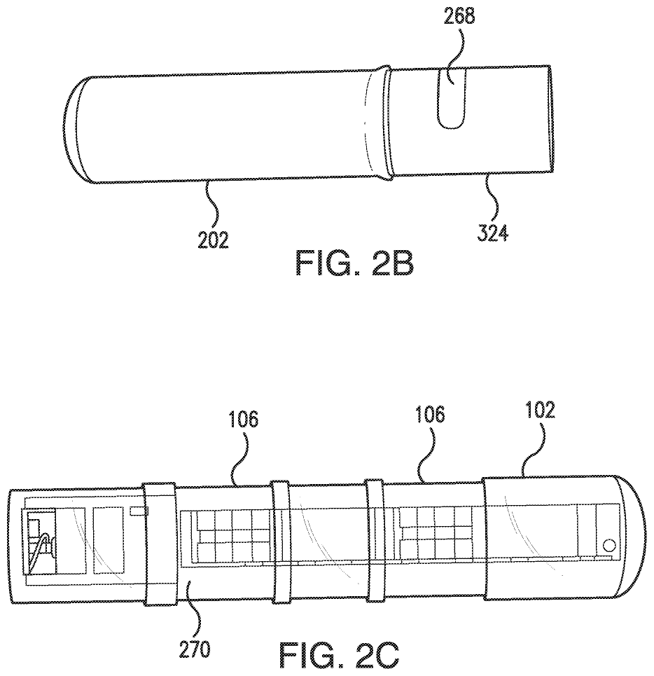

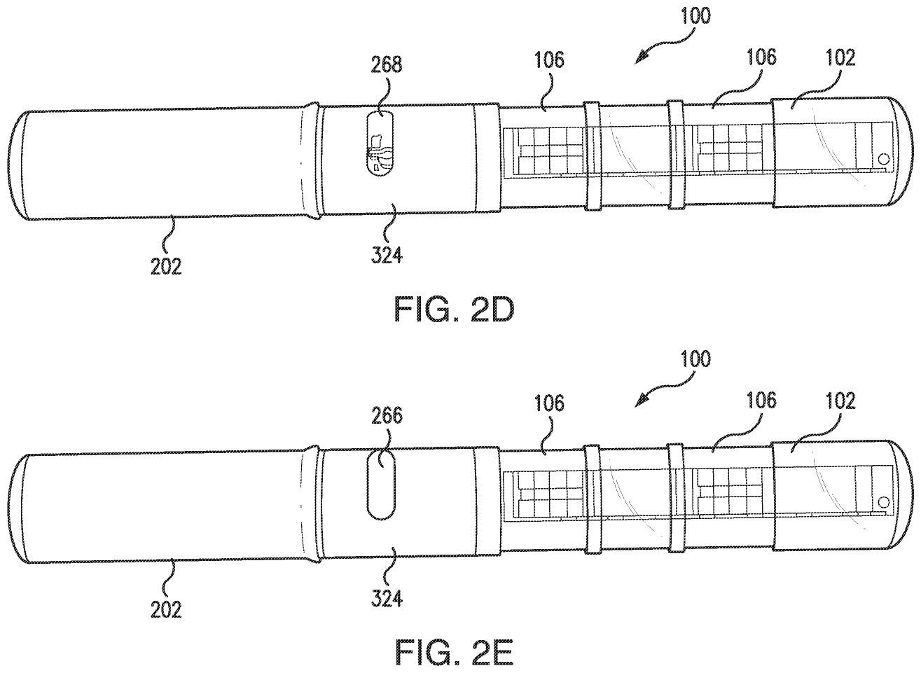

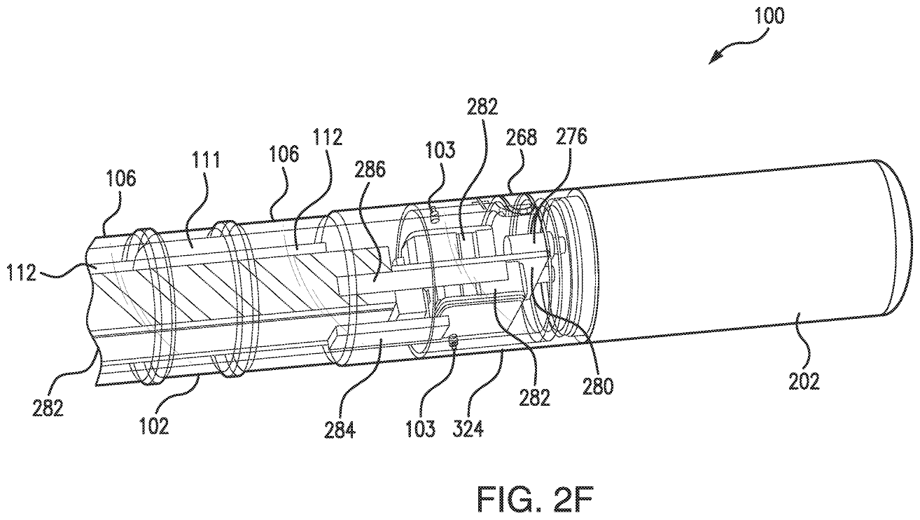

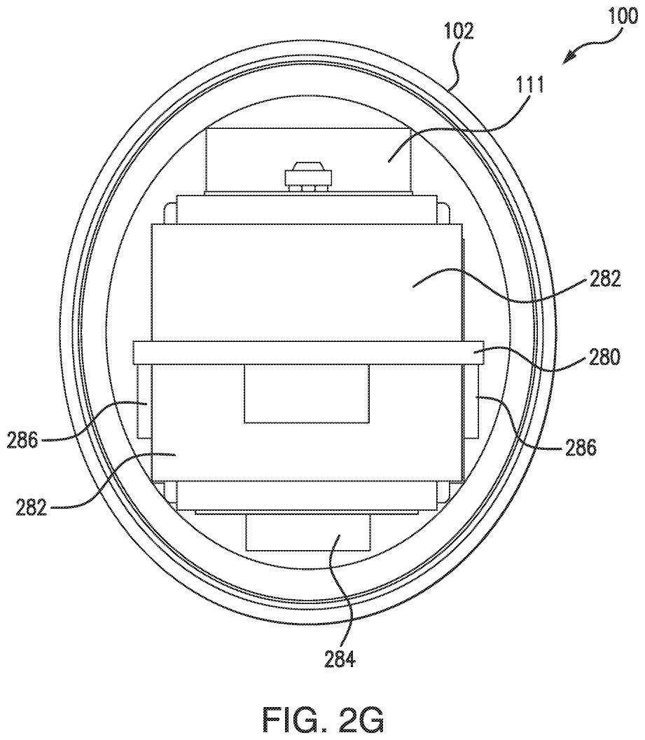

FIG. 2A is an exploded view of the apparatus 100 of the system 50 according to some aspects. In some aspects, as shown in FIG. 2A, the apparatus 100 may include a housing 102, circuitry 270, a power source 202, first and second electrically conductive leads 276 and 278, and/or a coupler 324. In some aspects, as shown in FIG. 2B, a first end of the coupler 324 may be attached to the power source 202. In some aspects, as shown in FIG. 2C, the circuitry 270 may be at least partially within the housing 102. In some aspects, as shown in FIGS. 2D and 2E, at least a portion of the housing 102 may extend into a second end of the coupler 324.

In some aspects, the housing 102 may be a body, shell, capsule, sleeve, or tube. In some aspects, the housing 208 may be rigid and/or biocompatible. In some aspect, the housing 208 may include a polymer (e.g., PMMA or silicone). However, this is not required, and, in other aspects, different materials and/or shapes may be used for the housing 208.

In some aspects, the apparatus 100 may include one or more analyte indicators 106, which may be, for example, polymer grafts or hydrogels coated, diffused, adhered, embedded, or grown on or in one or more portions of the exterior surface of the housing 102. In some aspects, as shown in FIGS. 2A, 2C, 2D, and 2E, the housing 102 may include one or more cutouts or recesses, and one or more analyte indicators 106 may be located (partially or entirely) in the cutouts or recesses. In some aspects, the one or more analyte indicators 106 may be porous and may allow an analyte (e.g., glucose) in a medium (e.g., interstitial fluid) to diffuse into the one or more analyte indicators 106.

In some aspects, the one or more analyte indicators 106 may have one or more detectable properties (e.g., optical properties) that vary in accordance with the amount or concentration of the analyte in proximity to the indicator element 106. In some aspects, the one or more analyte indicators 106 may include one or more analyte indicator molecules (e.g., fluorescent analyte indicator molecules), which may be distributed throughout the one or more analyte indicators 106. In some aspects, the one or more analyte indicators 106 may be phenylboronic-based analyte indicators. However, a phenylboronic-based analyte indicator is not required, and, in some alternative aspects, the one or more analyte indicators 106 may be different analyte indicators, such as, for example and without limitation, glucose oxidase-based indicators, glucose dehydrogenase-based indicators, and glucose binding protein-based indicators.

In some aspects, the circuitry 270 may include measurement electronics (e.g., optical measurement electronics), one or more circuit components 111 (e.g., analog and/or digital circuit components), an antenna 114, one or more capacitors 282, and/or first and second contact pads 272 and 274. In some aspects, the measurement electronics of the circuitry 270 may include one or more light sources 108 (e.g., one or more light emitting diodes (LEDs)) and one or more photodetectors 224 (e.g., one or more photodiodes, phototransistors, photoresistors, or other photosensitive elements). In some aspects, the one or more light sources 108 may be configured to emit excitation light (e.g., ultraviolet (UV) light) that reaches the one or more analyte indicators 106. In some aspects, the one or more photodetectors 224 may be configured to detect emission light (e.g., fluorescent light) that reaches the one or more photodetectors 224 after being emitted by the one or more analyte indicators 106. In some aspects, the amount of emission light emitted by the one or more analyte indicators 106 may correspond to the amount of analyte (e.g., glucose) in the medium (e.g., interstitial fluid) in proximity to the one or more analyte indicators 106. For example, in some aspects, the analyte may bind reversibly to analyte indicator molecules of the one or more analyte indicators 106, analyte indicator molecules to which the analyte is bound may emit emission light when irradiated by the excitation light, and analyte indicator molecules to which the analyte is not bound may not emit light (or emit only a small amount of light) when irradiated by the excitation light.

In some aspects, as shown in FIG. 2A, the apparatus 100 may include one or more substrates 112. In some aspects, the one or more substrates 112 may be circuit boards (e.g., one or more flexible and/or rigid printed circuit boards (PCBs)). In some aspects, one or more of the circuit components 111 may be mounted or otherwise attached to the one or more substrates 112. However, in some alternative aspects, the one or more substrates 112 may be semiconductor substrates having one or more of the circuit components 111 fabricated therein. For instance, the fabricated circuit components may include analog and/or digital circuitry. Also, in some aspects in which the substrate 112 is a semiconductor substrate, in addition to the one or more circuit components fabricated in the semiconductor substrate, one or more circuit components may be mounted or otherwise attached to the semiconductor substrate. In other words, in some semiconductor substrate aspects, a portion or all of the circuit components 111, which may include discrete circuit elements, an integrated circuit (e.g., an application specific integrated circuit (ASIC)) and/or other electronic components (e.g., a non-volatile memory), may be fabricated in the semiconductor substrate with the remainder of the circuit components 111 secured to the semiconductor substrate, which may provide communication paths between the various secured components.

In some aspects, as shown in FIG. 2A, the measurement electronics of the circuitry 270 may be mounted on and/or fabricated in the one or more substrates 112. In some aspects, as shown in FIG. 2A, the one or more substrates 112 may include (i) a first set of one or more light sources 108 and one or more photodetectors 224 and (ii) a second set of one or more light sources 108 and one or more photodetectors 224. In some aspects, the one or more light sources 108 may be mounted on the one or more substrates 112, the one or more photodetectors 224 may be fabricated in the substrate 112, and all or a portion of the circuit components 111 may be fabricated within the substrate 112.

In some aspects, as shown in FIG. 2A, the antenna 114 may be an inductor including a conductor 702 in the form of a coil and a magnetic core 704. In some aspects, the core 704 may be, for example and without limitation, a ferrite core. In some aspects, the antenna 114 may be, for example, a ferrite-based micro-antenna. In some aspects, as illustrated in FIG. 2A, the one or more substrates 112 of the apparatus 100 may be attached to the antenna 114. In some aspects, the circuit components 111 of the substrates 112 may be connected electrically to the antenna 114. In some aspects, the apparatus 100 may use the antenna 114 to communicate data (e.g., measurement data) to the external device 101 and/or the display device 105. In some aspects, the apparatus 100 may use the antenna 114 for NFC.

In some aspects, as shown in FIG. 2A, the apparatus 100 may include a PCB 280. In some aspects, the one or more capacitors 282 of the circuitry 270 may be mounted on the PCB 280. In some aspects, the PCB 280 may include the first and second contact pads 272 and 274 of the circuitry 270. In some aspects, the circuit components 111 of the substrates 112 and/or the antenna 114 may be connected electrically to the one or more capacitors 282 and/or the first and second contact pads 272 and 274.

In some aspects, the apparatus 100 (e.g., the circuitry 270 of the apparatus 100) may be powered at least partially by the power source 202. In some aspects, the power source 202 may be a charge storage device (e.g., a battery, capacitor, or super capacitor). In some aspects, at least the exterior of the power source 202 may be made of a biocompatible material such as, for example and without limitation, stainless steel or a titanium alloy. In some aspects, the power source 202 may be a titanium-cased, hermetically-sealed battery. In some aspects, as shown in FIGS. 2A, 2D, and 2E, the circuitry 270 of the apparatus 100 may extend away from the power source 202 along the longitudinal axis of the power source 202.

In some aspects, the power source 202 may include first and second terminals (e.g., a positive terminal (cathode) and a negative terminal (anode)). In some aspects, the first and second electrically conductive leads 276 and 278 may be connected electrically to the first and second terminals, respectively, of the power source 202. In some aspects, the electrically conductive leads 276 and 278 may electrically connect the first and second terminals, respectively, of the power source 202 to the circuitry 270 of the apparatus 100. In some aspects, the electrically conductive leads 276 and 278 may be rods or beams including or made out of a conductive material.

In some aspects, as shown in FIGS. 2A, 2B, 2D, and 2E, the coupler 324 may be a flange. In some aspects, as shown in FIGS. 2B, 2D, and 2E, the coupler 324 may be attached to the power source 202. In some aspects, the coupler 324 may be welded (e.g., laser welded) to the power source 202. In some aspects, the coupler 324 may enclose the first and second terminals of the power source 202. In some aspects, as shown in FIGS. 2D and 2E, the coupler 324 may be between the housing 102 and the power source 202. In some aspects, as shown in FIG. 2E, the apparatus 100 may further include a cap 266 over the one or more openings 268 of the coupler 324.

In some aspects, the coupler 324 may have a generally cylindrical shape. However, other shapes (e.g., a generally rectangular prism shape) may be used in alternative aspects. In some aspects, the coupler 324 may be made of a biocompatible material such as, for example and without limitation, glass, ceramic, stainless steel, titanium, or a titanium alloy. In some aspects, the coupler 324 may include a flat surface that abuts and is attached to the power source 202.

In some aspects, as shown in FIGS. 2A, 2B, 2D, and 2E, the coupler 324 may include one or more openings 268 through which the first and second electrically conductive leads 276 and 278 are capable of being laser welded to the first and second contact pads 272 and 274, respectively, of the circuitry 270. In some aspects, the housing 102 may include one or more openings 103 through which the first and second electrically conductive leads 276 and 278 are capable of being laser welded to the first and second contact pads 272 and 274, respectively, of the circuitry 270.

In some aspects, the apparatus 100 may further include an encasement material that encases at least a first portion of the circuitry 270 in the housing 102. In some aspects, the first portion of the circuitry 270 may include the one or more light sources 108 and the one or more photodetectors 224. In some aspects, the encasement material may include a water-resistant epoxy.

In some aspects, the encasement material may be a first encasement material that encases the first portion of the circuitry 270, and the first portion of the circuitry may not include the first and second contact pads 272 and 274. In some aspects, the apparatus 100 may further include a second encasement material that encases the first and second electrically conductive leads 276 and 278 and a second portion of the circuitry 270. In some aspects, the second portion of the circuitry may include the first and second contact pads 272 and 274. In some aspects, the first and second encasement materials may be different. In some alternative aspects, the first and second encasement materials may be the same. In some aspects, the second encasement material may include a water-resistant epoxy. In some aspects, the first encasement material may fill a first portion of the housing 102, and the second encasement material may fill the coupler 324 and a second portion of the housing 102 that is not filled by the first encasement material. In some alternative aspects, instead of first and second encasement materials, the encasement material a single encasement material that encases the circuitry 270 and the first and second electrically conductive leads 276 and 278. In some aspects, the encasement material may fill the housing 102 and the coupler 324.

In some aspects, the excitation light emitted by the one or more light sources 108 of the circuitry 270 may reach the one or more analyte indicators 106 after passing through the encasement material (e.g., the first encasement material or the single encasement material). In some aspects, the emission light emitted by the one or more analyte indicators 106 may reach the one or more photodetectors 224 after passing through the encasement material (e.g., the first encasement material or the single encasement material).

In some aspects, as shown in FIGS. 2F and 2G, the apparatus 100 may include one or more supports. In some aspects, the one or more supports may stiffen the circuitry 270 including the antenna 114 and the PCB 280. In some aspects, as shown in FIGS. 2F and 2G, the apparatus 100 may include a bottom support 284. In some aspects, the bottom support 284 may be attached to and extending from a bottom surface of the antenna 114. In some aspects, the bottom support 284 may be attached to one or more of the capacitors 282 mounted on a bottom surface of the PCB 280. In some aspects, the bottom support 284 may be attached to the bottom surface of the antenna 114 and to the one or more of the capacitors 282 mounted on a bottom surface of the PCB 280 using an adhesive (e.g., an epoxy adhesive).

In some aspects, as shown in FIGS. 2F and 2G, the apparatus 100 may additionally or alternatively include side supports 286. In some aspects, the side supports 286 may be attached to and extending from side surfaces of the antenna 114. In some aspects, the side supports 286 may include at least one side support 286 attached to and extending from a right side surface of the antenna 114 and at least one side support 286 attached to and extending from a left side surface of the antenna 114. In some aspects, the side supports 286 may be attached to a bottom surface of the PCB 280. In some aspects, the side supports 286 may additionally or alternatively be attached to one or more of the capacitors 282 mounted on a bottom surface of the PCB 280. In some aspects, the side supports 286 may be attached to the side surfaces of the antenna 114, the bottom surface of the PCB 280, and/or the one or more of the capacitors 282 mounted on the bottom surface of the PCB 280 using an adhesive (e.g., an epoxy adhesive).

In some aspects, although not shown in FIGS. 2F and 2G, the apparatus 100 may additionally or alternatively include a top support. In some aspects, the top support may be attached to and extending from a top surface of the antenna 114. In some aspects, the top support may be attached to one or more of the capacitors 282 mounted on a top surface of the PCB 280. In some aspects, the top support may be attached to the top surface of the antenna 114 and the one or more of the capacitors 282 mounted on the top surface of the PCB 280 using an adhesive (e.g., an epoxy adhesive).

In some aspects, one or more of the stiffeners (e.g., one or more of the side supports 286 and/or the bottom support 284) may be made from a ceramic material. However, this is not required, and, in some alternative aspects, one or more of the stiffeners may be made from a different material. For example, in some aspects, one or more of the stiffeners may be a stiff, electrically insulative polymer (Polyphenylsulfone or Polyether-ether-ketone).

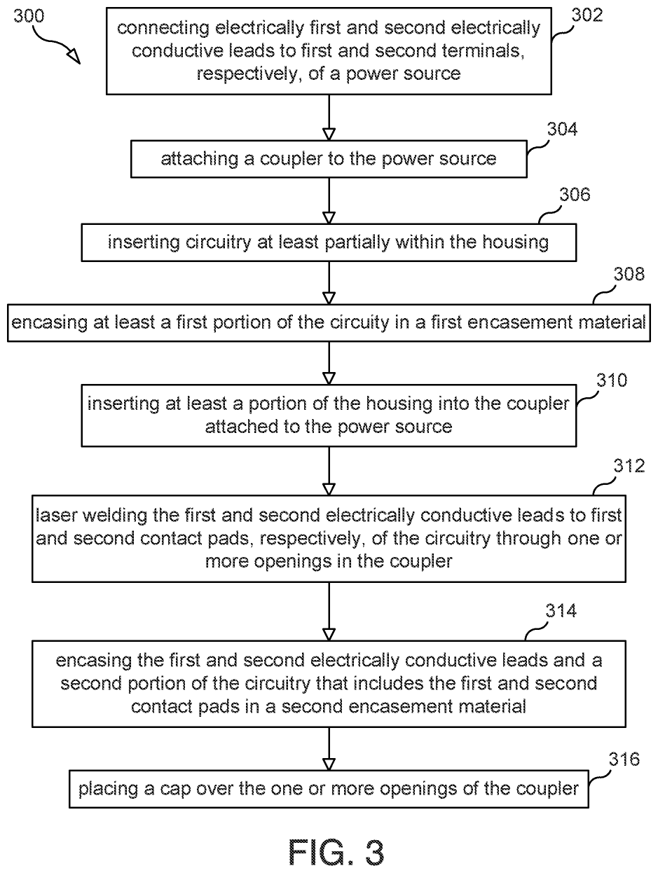

FIG. 3 illustrates a process 300 for manufacturing the apparatus 100 according to some aspects. In some aspects, the process 300 may include a step 302 of connecting electrically the first and second electrically conductive leads 276 and 278 to the first and second terminals, respectively, of the power source 202. In some aspects, connecting electrically the first and second electrically conductive leads 276 and 278 to the first and second terminals, respectively, of the power source 202 may include welding the first and second electrically conductive leads 276 and 278 to the first and second terminals, respectively, of the power source 202.

In some aspects, the process 300 may include a step 304 of attaching the coupler 324 (e.g., a first end of the coupler 324) to the power source 202. In some aspects, attaching a first end of the coupler 324 to the power source 202 is by, for example and without limitation, laser welding the first end of the coupler 324 to the power source 202.

In some aspects, the process 300 may include a step 306 of inserting the circuitry 270 at least partially within the housing 102.

In some aspects, the process 300 may include a step 308 of encasing at least a first portion of the circuitry 270 in a first encasement material. In some aspects, the first portion of the circuitry 270 may include the one or more light sources 108 and the one or more photodetectors 224. In some aspects, the first portion of the circuitry 270 may not include the first and second contact pads 272 and 274. In some aspects, the step 308 may include, after placing the circuitry 270 at least partially within the housing 102, filling the housing 102 with the first encasement material to an initial epoxy fill line. In some aspects, the initial epoxy fill line may be such that the first and second contact pads 272 and 274 are not exposed and not covered by the first encasement material. In some aspects, the step 308 may include curing the first encasement material. In some aspects, the cured first encasement material may create a transmissive optical cavity within the housing 102. In some aspects, the transmissive optical cavity may be formed from a suitable, optically transmissive polymer material. In some aspects, the first encasement material may additionally or alternatively include a water-resistant epoxy.

In some aspects, the process 300 may include a step 310 of inserting at least a portion of the housing 102 into the coupler 324, a first end of which may be attached to the power source 202. In some aspects, the housing 102 may be inserted into a second end of the coupler 324, which may be opposite the first end of the coupler 324.

In some aspects, the process 300 may include a step 312 of laser welding the first and second electrically conductive leads 276 and 278 to the first and second contact pads 272 and 274, respectively, of the circuitry 270 through the one or more openings 268 in the coupler 324. In some aspects, the laser welding of the first and second electrically conductive leads 276 and 278 to the first and second contact pads 272 and 274, respectively, of the circuitry 270 may be through the one or more openings 268 in the coupler 324 and the one or more openings 103 in the housing 102. In some aspects, the first and second electrically conductive leads 276 and 278 may be laser welded to the first and second contact pads 272 and 274, respectively, of the circuitry 270 by soldering bonding wires, which may be attached to ends of the first and second electrically conductive leads 276 and 278, to the contact pads 236.

In some aspects, the process 300 may include a step 314 of, after laser welding the first and second electrically conductive leads 276 and 278 to the first and second contact pads 272 and 274, respectively, of the circuitry 270 in the step 312, encasing the first and second electrically conductive leads 276 and 278 and a second portion of the circuitry 270 that includes the first and second contact pads 272 and 274 in a second encasement material. In some aspects, the second encasement material may fill the coupler 324 and a second portion of the housing 102 that was not filled by the first encasement material. In some aspects, the step 314 may include curing the second encasement material. In some aspects, the first and second encasement materials may be different. In some alternative aspects, the first and second encasement materials may be the same. In some aspects, the second encasement material may include a water-resistant epoxy.

In some aspects, encasing the first and second electrically conductive leads 276 and 278 and the second portion of the circuitry 270 in the second encasement material in step 314 may include inserting the second encasement material through the one or more openings 268 in the coupler 324. In some aspects, encasing the first and second electrically conductive leads 276 and 278 and the second portion of the circuitry 270 in the second encasement material in step 314 may further include inserting the second encasement material through the one or more openings 103 in the housing 102.

In some aspects, the process 300 may include a step 316 of, after laser welding the first and second electrically conductive leads 276 and 278 to the first and second contact pads 272 and 274 in step 314, placing the cap 266 over the one or more openings 268 of the coupler 324.

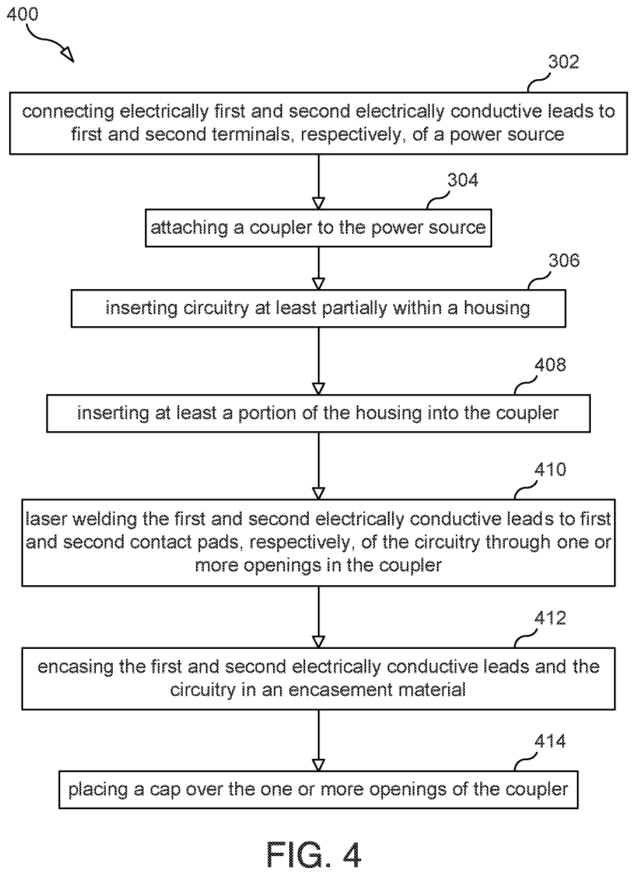

FIG. 4 illustrates a process 400 for manufacturing the apparatus 100 according to some alternative aspects. In some aspects, the process 400 may include the step 302 of connecting electrically the first and second electrically conductive leads 276 and 278 to the first and second terminals, respectively, of the power source 202. In some aspects, the process 400 may include the step 304 of attaching the coupler 324 to the power source 202. In some aspects, the process 400 may include the step 306 of inserting the circuitry 270 at least partially within the housing 102.

In some aspects, the process 400 may include a step 408 of inserting at least a portion of the housing 102 into the coupler 324, a first end of which may be attached to the power source 202. In some aspects, the housing 102 may be inserted into a second end of the coupler 324, which may be opposite the first end of the coupler 324.

In some aspects, the process 400 may include a step 410 of laser welding the first and second electrically conductive leads 276 and 278 to the first and second contact pads 272 and 274, respectively, of the circuitry 270 through the one or more openings 268 in the coupler 324. In some aspects, the laser welding of the first and second electrically conductive leads 276 and 278 to the first and second contact pads 272 and 274, respectively, of the circuitry 270 may be through the one or more openings 268 in the coupler 324 and the one or more openings 103 in the housing 102. In some aspects, the first and second electrically conductive leads 276 and 278 may be laser welded to the first and second contact pads 272 and 274, respectively, of the circuitry 270 by soldering bonding wires, which may be attached to ends of the first and second electrically conductive leads 276 and 278, to the contact pads 236.

In some aspects, the process 400 may include a step 412 of, after laser welding the first and second electrically conductive leads 276 and 278 to the first and second contact pads 272 and 274, respectively, of the circuitry 270 in the step 410, encasing the circuitry 270 and the first and second electrically conductive leads 276 and 278 in an encasement material. In some aspects, the encasement material may fill the coupler 324 and the housing 102. In some aspects, encasing the circuitry 270 and the first and second electrically conductive leads 276 and 278 in the encasement material may include inserting the encasement material through the one or more openings 268 in the coupler 324. In some aspects, encasing the circuitry 270 and the first and second electrically conductive leads 276 and 278 in the encasement material may further include inserting the encasement material through one or more openings 103 in the housing 102.

In some aspects, the step 412 may include curing the encasement material. In some aspects, the cured encasement material may create a transmissive optical cavity within the housing 102. In some aspects, the transmissive optical cavity may be formed from a suitable, optically transmissive polymer material. In some aspects, the encasement material may include a water-resistant epoxy.

In some aspects, the process 400 may include a step 414 of, after laser welding the first and second electrically conductive leads 276 and 278 to the first and second contact pads 272 and 274 in step 412, placing the cap 266 over the one or more openings 268 of the coupler 324.

Aspects of the present invention have been fully described above with reference to the drawing figures. Although the invention has been described based upon these preferred aspects, it would be apparent to those of skill in the art that certain modifications, variations, and alternative constructions could be made to the described aspects within the spirit and scope of the invention. For example, in some aspects, the apparatus 100 may include a bridging material with insulation.

1. An apparatus comprising:
a housing;
circuitry at least partially within the housing, wherein the circuitry comprises first and second contact pads;
a power source including first and second terminals;
first and second electrically conductive leads connected electrically to the first and second terminals, respectively, of the power source; and
a coupler attached to the power source, wherein at least a portion of the housing extends into the coupler, and the coupler comprises one or more openings through which the first and second electrically conductive leads are capable of being laser welded to the first and second contact pads, respectively, of the circuitry.

2. The apparatus of claim 1, wherein the housing comprises one or more openings through which the first and second electrically conductive leads are capable of being laser welded to the first and second contact pads, respectively, of the circuitry.

3. The apparatus of claim 1, wherein the housing is a polymethylmethacrylate (PMMA) housing.

4. The apparatus of claim 1, wherein the housing is a sleeve.

5. The apparatus of claim 1, wherein the power source is a battery.

6. The apparatus of claim 5, wherein the battery is a titanium-cased, hermetically-sealed battery.

7. The apparatus of claim 1, wherein the coupler covers the first and second terminals of the power source.

8. The apparatus of claim 1, wherein the coupler comprises titanium.

9. The apparatus of claim 1, further comprising an encasement material that encases at least a first portion of the circuitry.

10. The apparatus of claim 9, wherein the encasement material comprises a water-resistant epoxy.

11. The apparatus of claim 9, wherein the encasement material is a first encasement material that encases the first portion of the circuitry, the first portion of the circuitry does not include the first and second contact pads, and the apparatus further comprises a second encasement material that encases the first and second electrically conductive leads and a second portion of the circuitry that includes the first and second contact pads.

12. The apparatus of claim 11, wherein the first and second encasement materials are different.

13. The apparatus of claim 11, wherein the first and second encasement materials are the same.

14. The apparatus of claim 11, wherein the second encasement material comprises a water-resistant epoxy.

15. The apparatus of claim 11, wherein the first encasement material fills a first portion of the housing, and the second encasement material fills the coupler and a second portion of the housing that is not filled by the first encasement material.

16. The apparatus of claim 9, wherein the encasement material encases the circuitry and the first and second electrically conductive leads.

17. The apparatus of claim 9, wherein the encasement material fills the housing and the coupler.

18. The apparatus of claim 9, further comprising one or more analyte indicators that cover one or more portions of an exterior surface of the housing.

19. The apparatus of claim 18, wherein the circuitry comprises one or more light sources configured to emit excitation light that reaches the one or more analyte indicators after passing through the encasement material.

20. The apparatus of claim 18, wherein the circuitry comprises one or more photodetectors configured to detect emission light that reaches the one or more photodetectors after being emitted by the one or more analyte indicators and passing through the encasement material.

21. The apparatus of claim 1, further comprising a cap over the one or more openings of the coupler.

22. A method comprising:
inserting at least a portion of a housing into a coupler attached to a power source, wherein circuitry is at least partially within the housing, the circuitry comprises first and second contact pads, and the power source includes first and second terminals; and
laser welding first and second electrically conductive leads to the first and second contact pads, respectively, of the circuitry through one or more openings in the coupler, wherein the first and second electrically conductive leads are connected electrically to the first and second terminals, respectively, of the power source.

23. The method of claim 22, wherein the housing comprises one or more openings, and the laser welding of the first and second electrically conductive leads to the first and second contact pads, respectively, of the circuitry is through the one or more openings in the coupler and the one or more openings in the housing.

24. The method of claim 22, further comprising, before inserting at least the portion of the housing into the coupler, encasing at least a first portion of the circuitry in a first encasement material.

25. The method of claim 24, wherein the first encasement material comprises a water-resistant epoxy.

26. The method of claim 24, wherein the first portion of the circuitry does not include the first and second contact pads, and the method further comprises, after laser welding the first and second electrically conductive leads to the first and second contact pads, respectively, of the circuitry, encasing the first and second electrically conductive leads and a second portion of the circuitry that includes the first and second contact pads in a second encasement material.

27. The method of claim 26, wherein the second encasement material fills the coupler and a second portion of the housing that was not filled by the first encasement material.

28. The method of claim 26, wherein the first and second encasement materials are different.

29. The method of claim 26, wherein the first and second encasement materials are the same.

30. The method of claim 26, wherein the second encasement material comprises a water-resistant epoxy.

31. The method of claim 26, wherein encasing the first and second electrically conductive leads and the second portion of the circuitry in the second encasement material comprises inserting the second encasement material through the one or more openings in the coupler.

32. The method of claim 31, wherein encasing the first and second electrically conductive leads and the second portion of the circuitry in the second encasement material further comprises inserting the second encasement material through one or more openings in the housing.

33. The method of claim 22, further comprising, after laser welding the first and second electrically conductive leads to the first and second contact pads, respectively, of the circuitry, encasing the circuitry and the first and second electrically conductive leads in an encasement material.

34. The method of claim 33, wherein the encasement material comprises a water-resistant epoxy.

35. The method of claim 33, wherein the encasement material fills the coupler and the housing.

36. The method of claim 33, wherein encasing the circuitry and the first and second electrically conductive leads in the encasement material comprises inserting the encasement material through the one or more openings in the coupler.

37. The method of claim 36, wherein encasing the circuitry and the first and second electrically conductive leads in the encasement material further comprises inserting the encasement material through one or more openings in the housing.

38. The method of claim 22, further comprising, after laser welding the first and second electrically conductive leads to the first and second contact pads, placing a cap over the one or more openings of the coupler.

39. A coupler comprising:
a first end configured to be attached to a power source;
a second end configured for insertion of at least a portion of a housing into the coupler; and
one or more openings through which first and second electrically conductive leads connected electrically to first and second terminals, respectively, of the power source are capable of being laser welded to first and second contact pads, respectively, of circuitry that is at least partially within the housing.

40. The coupler of claim 39, wherein the coupler is configured to cover the first and second terminals of the power source.

41. The coupler of claim 39, wherein the coupler comprises titanium.

42. An apparatus comprising:
a housing;
circuitry at least partially within the housing, wherein the circuitry includes an antenna and a printed circuit board (PCB); and
one or more supports attached to and extending from the antenna and configured to stiffen the circuitry.

43. The apparatus of claim 42, wherein the one or more supports include a bottom support that is attached to and extends from a bottom surface of the antenna.

44. The apparatus of claim 43, wherein the circuitry includes one or more capacitors mounted on a bottom surface of the PCB, and the bottom support is attached to the one or more capacitors mounted on the bottom surface of the PCB.

45. The apparatus of claim 42, wherein the one or more supports include side supports that are attached to and extend from side surfaces of the antenna.

46. The apparatus of claim 45, wherein the side supports include at least one side support attached to and extending from a right side surface of the antenna and at least one side support attached to and extending from a left side surface of the antenna.

47. The apparatus of claim 45, wherein the side supports are attached to a bottom surface of the PCB.

48. The apparatus of claim 45, wherein the circuitry includes one or more capacitors mounted on a bottom surface of the PCB, and the side supports are attached to the one or more capacitors mounted on the bottom surface of the PCB.

49. The apparatus of claim 42, wherein the one or more supports include a top support that is attached to and extends from a top surface of the antenna.

50. The apparatus of claim 49, wherein the circuitry includes one or more capacitors mounted on a top surface of the PCB, and the top support is attached to the one or more capacitors mounted on the top surface of the PCB.
